SEQUENCE LISTING (1 sequence)

SEQ ID NO: 1 - protein, 15 aa, Homo sapiens
AVLTQKQKKQSAAAC

(19) United States
(12) Patent Application Publication
(10) Pub. No.: US 2012/0201823 A1
(43) Pub. Date: Aug. 9, 2012

(54) APRIL ANTAGONISTS AND METHODS OF USE

(75) Inventors: Mary J. Janatpour, Castro Valley, CA (US); Kathy L. Miller, San Francisco, CA (US)

(73) Assignee: Schering Corporation, Rahway, NJ (US)

(21) Appl. No.: 13/501,702

(22) PCT Filed: Oct. 14, 2010
(86) PCT No.: PCT/US10/52614

§ 371 Date: Apr. 12, 2012

Related U.S. Application Data
(60) Provisional application No. 61/251,574, filed on Oct. 14, 2009.
(60) Provisional application No. 61/307,775, filed on Feb. 24, 2010.

(51) Int. Cl. A61K 39/395; A61P 37/06; A61P 35/00

(52) U.S. Cl. 424/136.1; 424/158.1; 424/133.1

(57) ABSTRACT

The present invention provides antagonists of APRIL. In particular, the APRIL antagonists block unique receptor binding sited on APRIL, BCMA. TACI, and/or HSPG. Also provided are methods of use.

### FIELD OF THE INVENTION

The present invention provides antibodies raised against APRIL, and methods for treating and diagnosing immune, proliferative, and hematopoietic conditions. In particular, it provides antibodies with dual specificities against distinct APRIL epitopes.

### BACKGROUND OF THE INVENTION

The tumor-necrosis factor (TNF)-related cytokines are mediators of host defense and immune regulation. Members of this family exist in membrane-anchored forms, acting locally through cell-to-cell contact, or as secreted proteins capable of diffusing to more distant targets. A parallel family of receptors signals the presence of these molecules leading to the initiation of cell death or cellular proliferation and differentiation in the target tissue. Presently, the TNF family of ligands and receptors has at least 13 recognized receptor-ligand pairs, including: TNF:TNF-R; LT-α:TNF-R; LT-α/β:LT-β-R; FasL:Fas; CD40L:CD40; CD30L:CD30; CD27L:CD27; OX40L:OX40 and 4-1BBL:4-1BB; trance/rankL: Light and Tweak.

APRIL (aka TNFSF13) is a member of the tumor necrosis factor (“TNF”) superfamily that induces both in vivo and in vitro B cell proliferation and differentiation (See e.g. U.S. Patent Application Nos. 60/016,812; 60/211,537; 60/241,952; 60/254,875; 60/277,978; and 08/815,783; and International Publication No. WO97/33902; and Yu et al., Nature Immunol. 1(3):252-256 (2000)). APRIL is distinguishable from other B cell growth and differentiation factors such as IL-2, IL-4, IL-5, IL-6, IL-7, IL-13, IL-15, CD40L, or CD27L (CD70) by its monocyte-specific gene and protein expression pattern and its specific receptor distribution and biological activity on B lymphocytes. APRIL expression is not detected in natural killer (“NK”) cells, T cells or B cells, but is restricted to cells of myeloid origin. The gene encoding APRIL has been mapped to chromosome 17p 13.

APRIL is expressed as a 250 amino acid type II membrane-bound polypeptide and a soluble 146 amino acid polypeptide. APRIL has an extracellular domain of 201 amino acids, a cytoplasmic domain of 28 amino acids, and one N-linked glycosylation site (see, e.g., Hahne et al. (1998) J. Exp. Med. 188:1185-1190; and Dillon et al. (2006) Nat. Rev. Drug. Disc. 5:235-246). APRIL is processed intracellularly in the Golgi apparatus by furin convertase to produce a biologically active secreted form (Lopez-Frag et al. (2001) EMBO reports 2:945-951). Soluble recombinant APRIL has been shown to induce in vitro proliferation of murine splenic B cells and to bind to a cell-surface receptor on these cells and also on T cells (Yu et al., (2000), supra). Soluble APRIL administration to mice has been shown to result in an increase in B cell numbers in the spleen and mesenteric lymph node, and an increase in serum IgM levels (Yu et al., (2000), supra).

APRIL is expressed by a number of cancer cell lines, both hematopoietic and epithelial in origin (see, Hahne, supra; He, et al. (2004) J. Immunol. 172:3268-3279; and Deshayes, et al. (2004) Oncogene 23:3005-3012). APRIL binds to at least three receptors, BCMA and TACI (both of which also bind BlyS), and heparin sulphate proteoglycans (HSPG-3). The receptors shared by APRIL and BlyS (BCMA, TACI) are expressed in lymphomas and multiple myelomas. HSPGs are widely expressed on epithelial and hematopoeitic cells. Epithelial cancers do not express BCMA or TACI, therefore APRIL binding to epithelial cancers, is through HSPG. APRIL mediates proliferation of epithelial cells through the HSPG engagement. The HSPG binding site on APRIL, near the N-terminus (see, e.g., Dillon, et al., supra), is distinct from the BCMA and TACI binding sites (see, e.g., Hendriks et al. (2005) Cell Death Dill: 12:637-648).

Presently, antagonists of APRIL can only be used to treat hematopoietic cancers. Thus a need exists to treat both hematopoietic and epithelial cancers. Furthermore, antagonizing both APRIL binding sites is likely to improve the efficacy in treating hematopoietic cancers. The present invention fulfills this need by providing bispecific antagonists of APRIL to treat various types of cancers, as well as autoimmune disorders.

### SUMMARY OF THE INVENTION

The present invention is based, in part, upon the discovery that antagonizing APRIL at binding sites for BCMA or TACI, and HSPG exhibits higher inhibition of APRIL surface binding and of hematopoietic cancer cell line proliferation than antagonizing each binding site individually. This bispecific antagonism may also be useful in the treatment of autoimmune disorders.

The present invention encompasses a method of inhibiting a cancer by administering at least one APRIL polypeptide antagonist, wherein the antagonist binds to at least two distinct epitopes on the APRIL polypeptide. In certain embodiments, the epitopes comprise a binding site for BCMA or TACI, and a binding site for HSPG. In other embodiments, a first and a second APRIL antagonist are administered. The first APRIL antagonist is an antibody or fragment thereof, raised against the binding site for BCMA or TACI; and the second APRIL antagonist is an antibody or fragment thereof, raised against the binding site for HSPG. Alternatively, the first APRIL antagonist comprises a soluble BCMA protein or a soluble TACI protein, and the second APRIL antagonist comprises an antibody or fragment thereof that binds to the HSPG binding site. In a further embodiments the APRIL antagonist is a bispecific antibody, or fragment thereof, that binds to the BCMA or TACI binding site and the HSPG binding site. The bispecific antibody or fragment thereof is a humanized or fully human antibody.

The present invention encompasses a method of inhibiting a B cell disorder by administering at least one APRIL antagonist, wherein the antagonist binds to at least two distinct epitopes on an APRIL polypeptide. The epitopes comprise a binding site for BCMA or TACI, and a binding site for HSPG. In certain embodiments, a first and a second APRIL antagonist are administered It is further contemplated that the first APRIL antagonist comprises an antibody or fragment thereof, is raised against the binding site for BCMA or TACI; and the second APRIL antagonist comprises an antibody or fragment thereof, raised against the binding site for HSPG. In certain embodiments, the first APRIL antagonist comprises a soluble BCMA protein or a soluble TACI protein, and the second APRIL antagonist comprises an antibody or fragment thereof that binds to the HSPG binding site. The APRIL antagonist can be a bispecific antibody, or fragment thereof, that binds to the BCMA or TACI binding site, and the HSPG binding site. In certain embodiments, the bispecific antibody or fragment thereof is a humanized or fully human antibody.

The present invention encompasses a method of inhibiting a cancer or B cell disorder by administering an APRIL antagonist, wherein the antagonist inhibits APRIL binding to HSPG. The APRIL antagonist binds to an epitope on APRIL comprising the binding site for HSPG.

In certain embodiments, the APRIL antagonist is an antibody or fragment thereof raised against the binding site for HSPG. The antibody or fragment thereof can be a humanized or fully human antibody.

### BRIEF DESCRIPTION OF THE DRAWINGS

FIG. 1 shows binding of 124 to the APRIL-Fc protein in a concentration-dependent manner.

FIG. 2 shows binding of 124 to the HSPG peptide at various concentrations.

FIG. 3 shows that the 124 mAb does not compete with BCMA binding of APRIL.

FIG. 4 shows that the polyclonal HSPG antibody blocks binding of 124 to APRIL.

FIG. 5 shows inhibition of Pfeiffer cell proliferation in the presence of 124, BCMA-Fc, or TACI-Fc.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### DEFINITIONS

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding composition with a target where the association results in reduction in the normal Brownian motion of the binding composition, in cases where the binding composition can be dissolved or suspended in solution. “Binding composition” refers to a binding compound in combination with a stabilizer, excipient, salt, buffer, solvent, or additive.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they retain, or are modified to comprise, a ligand-specific binding domain. The antibody herein is directed against an “antigen” of interest. Preferably, the antigen is a biologically important polypeptide and administration of the antibody to a mammal suffering from a disease or disorder can result in a therapeutic benefit in that mammal. However, antibodies directed against nonpolypeptide antigens (such as tumor-associated glycolipid antigens; see U.S. Pat. No. 5,091,178) are also contemplated. Where the antigen is a polypeptide, it may be a transmembrane molecule (e.g. receptor) or ligand such as a growth factor. Exemplary antigens include those polypeptides.

As used herein, the terms “binding fragment” or “antigen binding fragment” encompass a fragment or a derivative of an antibody that still substantially retains its biological activity, e.g. inhibiting cytokine signaling via the cytokine receptor. The term “antibody fragment” refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its inhibitory activity. Preferably, a binding fragment or derivative retains at least 25%, 50%, 60%, 70%, 80%, 90%, 95%, 99% or 100% (or more) of its inhibitory activity, although any binding fragment with sufficient affinity to exert the desired biological effect will be useful. It is also intended that an antibody binding fragment can include variants having conservative amino acid substitutions that do not substantially alter its biologic activity.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody (or “scFv antibody”) refers to antibody fragments comprising the V_{H} and V_{I}, domains of an antibody, wherein these domains are present in a single polypeptide chain. Generally, the Fv polypeptide further comprises a polypeptide linker between the V_{H} and V_{I}, domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also WO 88/01649 and U.S. Pat. Nos. 4,946,778 and 5,260,203. Such scFv polypeptides may optionally be joined with Fc regions to form scFv-Fc constructs. See, e.g., Powers et al. (2001) J. Immunol. Methods 251:123.

A “diabody” is a small antibody fragment with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

A “bivalent antibody” comprises two antigen binding sites. In some instances, the two binding sites have the same antigen specificities. However, bivalent antibodies may be bispecific (see below).

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

Bispecific antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368. Potentially bispecific antibody fragments include diabodies, Bis-scFv, bivalent domain antibody fragments, Fab₂, and even Fab₃ fragments (which may be trispecific) (see Holliger and Hudson (2005) Nat. Biotechnol. 23:1126) and Bis-scFv-Fc. Bispecific antibodies also include dual variable domain immunoglobulins, such as those disclosed at U.S. Patent Application Publication No. 2005/0071675.

The antibodies of the present invention also include antibodies or fragments thereof conjugated to cytotoxic payloads, such as cytotoxic agents or radionuclides. Such antibody conjugates may be used in immunotherapy to selectively target and kill cells expressing a target (the antigen for that antibody) on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See, e.g., U.S. Patent Application Publication No. 2006/0014225.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al, J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The humanized forms of rodent antibodies will generally comprise the same CDR sequences of the parental rodent antibodies, although certain amino acid substitutions may be included to increase affinity, increase stability of the humanized antibody, or for other reasons.

The term “antibody” also includes “fully human” antibodies, i.e., antibodies that comprise human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods. Also, recombinant immunoglobulins may also be made in transgenic mice. See Mendez et al. (1997) Nature Genetics 15:146-156. See also Abgenix and Medarex technologies.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in the a targeted cell.

The antibodies may also be conjugated (e.g., covalently linked) to molecules that improve stability of the antibody during storage or increase the half-life of the antibody in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g., Chapman (2002) Adv. Drug Deliv. Rev. 54:531-545; Anderson and Tomasi (1988) J. Immunol. Methods 109:37-42; Suzuki et al. (1984) Biochim. Biophys. Acta 788:248-255; and Brekke and Sandlie (2003) Nature Rev. 2:52-62.

Antibodies used in the present invention will usually bind with at least a K_{d} of about 10⁻³M, more usually at least 10⁻⁶M, typically at least 10⁻⁷M, more typically at least 10⁻⁸M, preferably at least about 10⁻⁹M, and more preferably at least 10⁻¹⁰ M, and most preferably at least 10⁻¹¹M. See, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample.

Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts a target, such as IL-23 receptor, e.g., in the fluid phase or colloidal phase, but also situations where the agonist or antagonist does not contact the cell or the receptor.

“Treat” or “Treating” may also refer to administration of a therapeutic agent, such as a composition described herein, internally or externally to a patient in need of the therapeutic agent. Typically, the agent is administered in an amount effective to prevent or alleviate one or more disease symptoms, or one or more adverse effects of treatment with a different therapeutic agent, whether by preventing the development of, inducing the regression of, or inhibiting the progression of such symptom(s) or adverse effect(s) by any clinically measurable degree. The amount of a therapeutic agent that is effective to alleviate any particular disease symptom or adverse effect (also referred to as the “therapeutically effective amount”) may vary according to factors such as the disease state, age, and weight of the patient, the ability of the therapeutic agent to elicit a desired response in the patient, the overall health of the patient, the method, route and dose of administration, and the severity of side affects. See, e.g., U.S. Pat. No. 5,888,530.

Whether a disease symptom or adverse effect has been alleviated can be assessed by any clinical measurement typically used by physicians or other skilled healthcare providers to assess the severity or progression status of that symptom or adverse effect. When a therapeutic agent is administered to a patient who has active disease, a therapeutically effective amount will typically result in a reduction of the measured symptom by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

While an embodiment of the present invention (e.g., a treatment method or article of manufacture) may not be effective in preventing or alleviating the target disease symptom(s) or adverse effect(s) in every patient, it should alleviate such symptom(s) or effect(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

An “antagonist,” as used herein, is any agent that reduces the activity of a targeted molecule. Specifically, an antagonist of a protein (such as APRIL) is an agent that reduces the biological activity of that protein, for example by blocking binding of the APRIL to TACI or BCMA, and/or HSPG or otherwise reducing its activity (e.g. as measured in a bioassay). As such, an antagonist includes any agent that reduces signaling, and thus may include agents that bind to the APRIL itself, and also agents that bind to its receptor(s). An antagonist further includes an agent that reduces the expression of APRIL or its receptor(s), including but not limited to nucleic acid-based antagonists, such as antisense nucleic acids and siRNA. See, e.g., Arenz and Schepers (2003) Naturwissenschaften 90:345-359; Sazani and Kole (2003) J. Clin. Invest. 112:481-486; Pirollo et al. (2003) Pharmacol. Therapeutics 99:55-77; Wang et al. (2003) Antisense Nud. Acid Drug Devel. 13:169-189.

Polypeptide antagonists include, but are not limited to, antagonistic antibodies, peptides, peptide-mimetics, polypeptides, and small molecules that bind to APRIL (or any of its subunits) or its functional receptor(s) in a manner that interferes with signal transduction and downstream activity. Examples of peptide and polypeptide antagonists include truncated versions or fragments of the BCMA or TACI receptor(s) (e.g., soluble extracellular domains) that bind to APRIL in a manner that interferes with APRIL binding to BCMA or TACI.

The terms “cancer”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are autoimmune diseases, immune-mediated inflammatory diseases, non-immune-mediated inflammatory diseases, infectious diseases, and immunodeficiency diseases. Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory and fibrotic lung diseases such as inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases include AIDS (HIV infection), hepatitis A, B, C, D, and E, bacterial infections, fungal infections, protozoal infections and parasitic infections.

A “B cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

Non-Hodgkin's lymphoma (NHL) includes, but is not limited to, low grade/follicular NHL, relapsed or refractory NHL, front line low grade NHL, Stage III/IV NHL, chemotherapy resistant NHL, small lymphocytic (SL) NHL, intermediate grade/follicular NHL, intermediate grade diffuse NHL, diffuse large cell lymphoma, aggressive NHL (including aggressive front-line NHL and aggressive relapsed NHL), NHL relapsing after or refractory to autologous stem cell transplantation, high grade immunoblastic NHL, high grade lymphoblastic NHL, high grade small non-cleaved cell NHL, bulky disease NHL, etc.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS)), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic throbocytopenic purpura (TTP), thrombocytopenia (as developed by myocardial infarction patients, for example), including autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barre Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

### General

The present invention provides methods of antagonizing APRIL activity by interfering with the binding of BCMA or TACI, and HSPG, to APRIL. APRIL has been shown to induce proliferation in a number of cancer cell lines, including: A549, Jurkat, Raji, HeLa, Me260 (Hahne et al. (1998) J Exp Med 188(6): 1185), as well as, HT29 and several glioblastoma lines (Deshayes et al. (2004) Oncogene 23:3005). In-house data suggests APRIL also enhances growth of the melanoma cell line MALME-3M, as well as other cancer lines. In addition to impacting growth, APRIL has been shown to mediate survival in B-cell lymphoma by inducing up-regulation of anti-apoptotic proteins, such as BCL2, BCL-xL and MCL-1. It has been demonstrated that NIH3T3-APRIL transfectants grow faster than control in vivo.

While the receptors in common to BlyS and APRIL are expressed in lymphoma and multiple myeloma, cancers that are epithelial in origin tend to be negative for both BCMA and TACI. In these cancer cells, BlyS has no activity, while APRIL both binds the surface and mediates proliferation. It has been shown that APRIL mediates its effects by binding HSPG (Hendriks et al. (2005) Cell Death Diff 12: 637) and that the interactions with HSPG are through a site distinct from that which binds BCMA and/or TACI.

### Generation of Epitope Specific APRIL Antibodies

Any suitable method for generating monoclonal antibodies may be used. For example, a recipient may be immunized with APRIL or a fragment thereof. Any suitable method of immunization can be used. Such methods can include adjuvants, other immuno stimulants, repeated booster immunizations, and the use of one or more immunization routes. Any suitable source of APRIL can be used as the immunogen for the generation of the non-human antibody of the compositions and methods disclosed herein. Such forms include, but are not limited to whole protein, peptide(s), and epitopes generated through recombinant, synthetic, chemical or enzymatic degradation means known in the art. In preferred embodiments the immunogen comprises the BCMA or TACI epitopes, and the HSPG epitope on APRIL.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Any suitable method can be used to elicit an antibody with the desired biologic properties to inhibit APRIL binding to BCMA or TACI and HSPG, or HSPG alone. It is desirable to prepare monoclonal antibodies (mAbs) from various mammalian hosts, such as mice, rats, other rodents, humans, other primates, etc. Description of techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a antigen binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. supra; and Ward et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance that provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. See also Abgenix and Medarex technologies.

Antibodies or binding compositions against predetermined fragments of APRIL (e.g. BCMA or TACI, and/or HSPG binding sites) can be raised by immunization of animals with conjugates of the polypeptide, fragments, peptides, or epitopes with carrier proteins. Monoclonal antibodies are prepared from cells secreting the desired antibody. These antibodies can be screened for binding to normal or defective APRIL. These monoclonal antibodies will usually bind with at least a K_{d} of about 1 μM, more usually at least about 300 nM, 30 nM, 10 nM, 3 nM, 1 nM, 300 pM, 100 pM, 30 pM or better, usually determined by ELISA.

### Humanization of APRIL Specific Antibodies

Any suitable non-human antibody can be used as a source for the hypervariable region. Sources for non-human antibodies include, but are not limited to, murine (e.g. Mus musculus), rat (e.g. Rattus norvegicus), Lagomorphs (including rabbits), bovine, and primates. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance of the desired biological activity. For further details, see Jones et al. (1986) Nature 321:522-525; Reichmann et al. (1988) Nature 332:323-329; and Presta (1992) Curr. Op. Struct. Biol. 2:593-596.

Methods for recombinantly engineering antibodies have been described, e.g., by Boss et al. (U.S. Pat. No. 4,816,397), Cabilly et al. (U.S. Pat. No. 4,816,567), Law et al. (European Patent Application Publication No. 438310) and Winter (European Patent Application Publication No. 239400).

Amino acid sequence variants of humanized anti-APRIL antibodies are prepared by introducing appropriate nucleotide changes into the humanized anti-APRIL antibodies' DNAs, or by peptide synthesis. Such variants include, for example, deletions from, and/or insertions into, and/or substitutions of, residues within the amino acid sequences shown for the humanized anti-APRIL antibodies. Any combination of deletion, insertion, and substitution is made to arrive at the final construct, provided that the final construct possesses the desired characteristics. The amino acid changes also may alter post-translational processes of the humanized anti-APRIL antibodies, such as changing the number or position of glycosylation sites.

A useful method for identification of certain residues or regions of the humanized anti-APRIL antibodies polypeptides that are preferred locations for mutagenesis is called “alanine scanning mutagenesis,” as described by Cunningham and Wells (1989) Science 244: 1081-1085. Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with APRIL antigen. The amino acid residues demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed humanized anti-APRIL antibodies' variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include humanized anti-APRIL antibodies with N-terminal methionyl residues or the antibodies fused to epitope tags. Other insertional variants of the humanized anti-APRIL antibody molecules include the fusion to the N- or C-termini of humanized anti-APRIL antibodies, of an enzyme or a polypeptide that increases the serum half-life of the antibody.

Another type of variant is an amino acid substitution variant. These variants have at least one amino acid residue in the humanized anti-APRIL antibody molecules removed and a different residue inserted in its place. The sites of greatest interest for substitutional mutagenesis include the hypervariable loops, but FR alterations are also contemplated.

Another type of amino acid variant of the antibody alters the original glycosylation pattern of the antibody. By altering is meant deleting one or more carbohydrate moieties found in the antibody, and/or adding one or more glycosylation sites that are not present in the antibody. Glycosylation of antibodies is typically either N-linked or O-linked N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Yet another type of amino acid variant is the substitution of residues to provide for greater chemical stability of the final humanized antibody. For example, an asparagine (N) residue may be changed to reduce the potential for formation of isoaspartate at any NG sequences within a rodent CDR. A similar problem may occur at a DG sequence. Reissner and Aswad (2003) Cell. Mol. Life. Sci. 60:1281. Isoaspartate formation may debilitate or completely abrogate binding of an antibody to its target antigen. Presta (2005) J. Allergy Clin. Immunol. 116:731 at 734. In one embodiment, the asparagine is changed to glutamine (Q). In addition, methionine residues in rodent CDRs may be changed to reduce the possibility that the methionine sulfur would oxidize, which could reduce antigen binding affinity and also contribute to molecular heterogeneity in the final antibody preparation. Id. In one embodiment, the methionine is changed to alanine (A). Antibodies with such substitutions are subsequently screened to ensure that the substitutions do not decrease APRIL binding affinity to unacceptable levels.

Nucleic acid molecules encoding amino acid sequence variants of humanized APRIL specific antibodies are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant versions of humanized anti-APRIL antibodies.

Ordinarily, amino acid sequence variants of the humanized anti-APRIL antibodies will have an amino acid sequence having at least 75% amino acid sequence identity with the original humanized antibody amino acid sequences of either the heavy or the light chain more preferably at least 80%, more preferably at least 85%, more preferably at least 90%, and most preferably at least 95%, 98% or 99%. Identity or homology with respect to this sequence is defined herein as the percentage of amino acid residues in the candidate sequence that are identical with the humanized residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

The humanized antibody can be selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Variants of the IgG isotypes are also contemplated. The humanized antibody may comprise sequences from more than one class or isotype. Optimization of the necessary constant domain sequences to generate the desired biologic activity is readily achieved by screening the antibodies in the biological assays described in the Examples.

Likewise, either class of light chain can be used in the compositions and methods herein. Specifically, kappa, lambda, or variants thereof are useful in the present compositions and methods.

Any suitable portion of the CDR sequences from the non-human antibody can be used. The CDR sequences can be mutagenized by substitution, insertion or deletion of at least one residue such that the CDR sequence is distinct from the human and non-human antibody sequence employed. It is contemplated that such mutations would be minimal. Typically, at least 75% of the humanized antibody residues will correspond to those of the non-human CDR residues, more often 90%, and most preferably greater than 95%.

Any suitable portion of the FR sequences from the human antibody can be used. The FR sequences can be mutagenized by substitution, insertion or deletion of at least one residue such that the FR sequence is distinct from the human and non-human antibody sequence employed. It is contemplated that such mutations would be minimal. Typically, at least 75% of the humanized antibody residues will correspond to those of the human FR residues, more often 90%, and most preferably greater than 95%, 98% or 99%. Also contemplated are chimeric antibodies. As noted above, typical chimeric antibodies comprise a portion of the heavy and/or light chain identical with, or homologous to, corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. See U.S. Pat. No. 4,816,567; and Morrison et al. (1984) Proc. Natl. Acad. Sci. USA 81: 6851-6855.

Bispecific antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

In yet other embodiments, different constant domains may be appended to humanized V_{L}, and V_{H} regions derived from the CDRs provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

The parental and engineered forms of the antibodies of the present invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al., (1999) (Bioconj. Chem. 10:973-981) discloses PEG conjugated single-chain antibodies. Wen et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments or the BCMA or TACI soluble proteins or fragments thereof of the invention may also be conjugated with labels such as ⁹⁹Tc, ⁹⁰ Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹ At, ²¹²Pb, ⁴⁷Sc, ¹⁰⁹Pd, ²³⁴Th and ⁴⁰K, ¹⁵⁷Gd, ⁵⁵Mn, ⁵²Tr and ⁵⁶Fe.

The antibodies and antibody fragments or the BCMA or TACT soluble proteins or fragments thereof of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., (1962) Nature 144:945; David et al., (1974) Biochemistry 13:1014; Pain et al., (1981) J. Immunol. Meth. 40:219; and Nygren, J., (1982) Histochem. and Cytochem. 30:407. Methods for conjugating antibodies and proteins are conventional and well known in the art.

### Immune an Proliferative Disorders

The present invention contemplates treatment of multiple cancers (melanoma, prostate cancer, colorectal cancer, multiple myeloma, lymphoma, etc.) with at least one APRIL antagonist, e.g., a bispecific antibody that blocks the ability of APRIL to bind HSPG and BCMA or TACI, and thus inhibit APRIL's activity in cancer cells. This will be superior to other proposed strategies, such as soluble BCMA-Ig or TACI-Ig, in that it will block APRIL's ability to bind epithelial cancer cells, and more effectively block APRIL's binding of cancer cells of myeloid or hematopoeitic origin.

This approach should also be useful in immune indications, such as autoimmune diseases. It is likely that an antagonist or antagonists targeting multiple unique binding sites on APRIL will likely be more effective in the treatment of B cell disorders than antagonizing BCMA or TACT binding alone (Sakurai, et al. (2007) Blood 109:2961-2967).

### Biological Activity of Humanized Anti-APRIL Antibodies

Antibodies having the characteristics identified herein as being desirable in humanized anti-APRIL antibodies can be screened for inhibitory biologic activity in vitro or suitable binding affinity. To screen for antibodies that bind to the BCMA or TACT, and/or HSPG epitopes on human APRIL bound by an antibody of interest (e.g., those that block binding of APRIL), a routine cross-blocking assay such as that described in Antibodies, A Laboratory Manual, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies bind at overlapping epitopes, or even nearby non-overlapping epitopes.

Alternatively, epitope mapping, e.g., as described in Champe et al. (1995) J. Biol. Chem. 270:1388-1394, can be performed to determine whether the antibody binds an epitope of interest. “Alanine scanning mutagenesis,” as described by Cunningham and Wells (1989) Science 244: 1081-1085, or some other form of point mutagenesis of amino acid residues in human APRIL may also be used to determine the functional epitope for anti-APRIL antibodies of the present invention. Mutagenesis studies, however, may also reveal amino acid residues that are crucial to the overall three-dimensional structure of APRIL but that are not directly involved in antibody-antigen contacts, and thus other methods may be necessary to confirm a functional epitope determined using this method.

The epitope bound by a specific antibody may also be determined by assessing binding of the antibody to peptides comprising fragments of human APRIL. A series of overlapping peptides encompassing the sequence of APRIL, specifically the BCMA, TACI, and/or HSPG epitopes, may be synthesized and screened for binding, e.g. in a direct ELISA, a competitive ELISA (where the peptide is assessed for its ability to prevent binding of an antibody to APRIL bound to a well of a microtiter plate), or on a chip. Such peptide screening methods may not be capable of detecting some discontinuous functional epitopes, i.e. functional epitopes that involve amino acid residues that are not contiguous along the primary sequence of the APRIL polypeptide chain.

The epitope bound by antibodies of the present invention may also be determined by structural methods, such as X-ray crystal structure determination (e.g., WO2005/044853), molecular modeling and nuclear magnetic resonance (NMR) spectroscopy, including NMR determination of the H-D exchange rates of labile amide hydrogens in APRIL when free and when bound in a complex with an antibody of interest (Zinn-Justin et al. (1992) Biochemistry 31:11335-11347; Zinn-Justin et al. (1993) Biochemistry 32:6884-6891).

With regard to X-ray crystallography, crystallization may be accomplished using any of the known methods in the art (e.g. Giege et al. (1994) Acta Crystallogr. D50:339-350; McPherson (1990) Eur. J. Biochem. 189:1-23), including microbatch (e.g. Chayen (1997) Structure 5:1269-1274), hanging-drop vapor diffusion (e.g. McPherson (1976) J. Biol. Chem. 251:6300-6303), seeding and dialysis. It is desirable to use a protein preparation having a concentration of at least about 1 mg/mL and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent, e.g. glycerol at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 3.0 to about 5.0, preferably about 4.0. Specific buffers useful in the precipitant solution may vary and are well-known in the art. Scopes, Protein Purification: Principles and Practice, Third ed., (1994) Springer-Verlag, New York. Examples of useful buffers include, but are not limited to, HEPES, Tris, MES and acetate. Crystals may be grow at a wide range of temperatures, including 2° C., 4° C., 8° C. and 26° C.

Antibody:antigen crystals may be studied using well-known X-ray diffraction techniques and may be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al. eds., Academic Press; U.S. Patent Application Publication No. 2004/0014194), and BUSTER (Bricogne (1993) Acta Cryst. D49:37-60; Bricogne (1997) Meth. Enzymol. 276A:361-423, Carter & Sweet, eds.; Roversi et al. (2000) Acta Cryst. D56:1313-1323).

Additional antibodies binding to the same epitope as an antibody of the present invention may be obtained, for example, by screening of antibodies raised against APRIL for binding to the epitope, or by immunization of an animal with a peptide comprising a fragment of human APRIL comprising the epitope sequences (e.g., BCMA or TACI, and/or HSPG epitopes). Antibodies that bind to the same functional epitope might be expected to exhibit similar biological activities, such as blocking receptor binding, and such activities can be confirmed by functional assays of the antibodies.

Antibody affinities may be determined using standard analysis. Preferred humanized antibodies are those that bind human APRIL with a K_{d} value of no more than about 1×10⁻⁷; preferably no more than about 1×10⁻⁸; more preferably no more than about 1×10⁻⁹; and most preferably no more than about 1×10⁻¹⁰ or even 1×10⁻¹¹ M.

The antibodies and fragments thereof useful in the present compositions and methods are biologically active antibodies and fragments. As used herein, the term “biologically active” refers to an antibody or antibody fragment that is capable of binding the desired the antigenic epitope and directly or indirectly exerting a biologic effect. As used herein, the term “specific” refers to the selective binding of the antibody to the target antigen epitope. Antibodies can be tested for specificity of binding by comparing binding to APRIL epitopes to binding to irrelevant antigen or antigen mixture under a given set of conditions. If the antibody binds to APRIL at least 10, and preferably 50 times more than to irrelevant antigen or antigen mixture then it is considered to be specific. An antibody that “specifically binds” to APRIL epitopes does not bind to proteins that do not comprise the APRIL-derived sequences, i.e. “specificity” as used herein relates to APRIL specificity, and not any other sequences that may be present in the protein in question. For example, as used herein, an antibody that “specifically binds” to a polypeptide comprising APRIL will typically bind to FLAG®-APRIL, which is a fusion protein comprising APRIL and a FLAG® peptide tag, but it does not bind to the FLAG® peptide tag alone or when it is fused to a protein other than APRIL.

APRIL-specific binding compounds of the present invention, such as antagonistic APRIL specific antibodies, can inhibit the binding of APRIL to its receptors, BCMA or TACI, and/or HSPG, and will be useful in the treatment of proliferative and immune disorders.

### Antibody Production

In one embodiment, for recombinant production of the antibodies of the present invention, the nucleic acids encoding the two chains are isolated and inserted into one or more replicable vectors for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence. In one embodiment, both the light and heavy chains of humanized anti-APRIL antibodies, or a bispecific APRIL antibody of the present invention are expressed from the same vector, e.g. a plasmid or an adenoviral vector.

Antibodies of the present invention may be produced by any method known in the art. In one embodiment, antibodies are expressed in mammalian or insect cells in culture, such as chinese hamster ovary (CHO) cells, human embryonic kidney (HEK) 293 cells, mouse myeloma NSO cells, baby hamster kidney (BHK) cells, Spodoptera frugiperda ovarian (Sf9) cells. In one embodiment, antibodies secreted from CHO cells are recovered and purified by standard chromatographic methods, such as protein A, cation exchange, anion exchange, hydrophobic interaction, and hydroxyapatite chromatography. Resulting antibodies are concentrated and stored in 20 mM sodium acetate, pH 5.5.

In another embodiment, the antibodies of the present invention are produced in yeast according to the methods described in WO2005/040395. Briefly, vectors encoding the individual light or heavy chains of an antibody of interest are introduced into different yeast haploid cells, e.g. different mating types of the yeast Pichia pastoris, which yeast haploid cells are optionally complementary auxotrophs. The transformed haploid yeast cells can then be mated or fused to give a diploid yeast cell capable of producing both the heavy and the light chains. The diploid strain is then able to secret the fully assembled and biologically active antibody. The relative expression levels of the two chains can be optimized, for example, by using vectors with different copy number, using transcriptional promoters of different strengths, or inducing expression from inducible promoters driving transcription of the genes encoding one or both chains.

In one embodiment, the respective heavy and light chains of a plurality of different anti-APRIL antibodies (the “original” antibodies) are introduced into yeast haploid cells to create a library of haploid yeast strains of one mating type expressing a plurality of light chains, and a library of haploid yeast strains of a different mating type expressing a plurality of heavy chains. These libraries of haploid strains can be mated (or fused as spheroplasts) to produce a series of diploid yeast cells expressing a combinatorial library of antibodies comprised of the various possible permutations of light and heavy chains. The combinatorial library of antibodies can then be screened to determine whether any of the antibodies has properties that are superior (e.g. higher affinity for APRIL) to those of the original antibodies. See. e.g., WO2005/040395.

In another embodiment, antibodies of the present invention are human domain antibodies in which portions of an antibody variable domain are linked in a polypeptide of molecular weight approximately 13 kDa. See, e.g., U.S. Pat. Publication No. 2004/0110941. Such single domain, low molecular weight agents provide numerous advantages in terms of ease of synthesis, stability, and route of administration.

### Combination Therapies

In addition to one or more APRIL antagonists, the present invention contemplates the co-administration of chemotherapeutic agents that include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON. toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

### Pharmaceutical Compositions and Administration

To prepare pharmaceutical or sterile compositions including APRIL antibodies or soluble BCMA or TACI proteins, the cytokine analogue or mutein, antibody thereto, or nucleic acid thereof, is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions. See, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration is not particularly important. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is substantially derived from the same species as the animal targeted for treatment (e.g. a humanized antibody for treatment of human subjects), thereby minimizing any immune response to the reagent.

Antibodies, antibody fragments, and cytokines can be provided by continuous infusion, or by doses at intervals of, e.g., one day, 1-7 times per week, one week, two weeks, monthly, bimonthly, etc. Doses may be provided intravenously, subcutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

As used herein, “inhibit” or “treat” or “treatment” includes a postponement of development of the symptoms associated with autoimmune disease or pathogen-induced immunopathology and/or a reduction in the severity of such symptoms that will or are expected to develop. The terms further include ameliorating existing uncontrolled or unwanted autoimmune-related or pathogen-induced immunopathology symptoms, preventing additional symptoms, and ameliorating or preventing the underlying causes of such symptoms. Thus, the terms denote that a beneficial result has been conferred on a vertebrate subject with an autoimmune or pathogen-induced immunopathology disease or symptom, or with the potential to develop such a disease or symptom.

As used herein, the term “therapeutically effective amount” or “effective amount” refers to an amount of an APRIL-specific binding compound, e.g. and antibody, that when administered alone or in combination with an additional therapeutic agent to a cell, tissue, or subject is effective to prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent, e.g., a cytokine, antibody, steroid, chemotherapeutic agent, antibiotic, or radiation, are well known in the art, see, e.g., Hardman et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA.

Typical veterinary, experimental, or research subjects include monkeys, dogs, cats, rats, mice, rabbits, guinea pigs, horses, and humans.

### EXAMPLES

### I. General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Cell Lines

PC3, HCT116, RPMI8226 and JVM2 cells were purchased from ATCC (American Type Culture Collection, MD). PC3, RPMI8226 and JVM2 cell lines were grown in RPMI 1640 (Mediatech Inc) with 10% fetal bovine serum (JRH 12103-78P). HCT116 cells were cultured in DMEM (Mediatech Inc 10-040-CV) with 10% fetal bovine serum. Cells were maintained at 37° C. in humidified air with 5% CO₂.

### III. siRNA Knockdown

One day before transfection, 1×10⁵ PC3 cells per 2.5 ml media were plated in each well of a 6 well plate. Cells were transfected with 100 nM SiRNA using Dharmafect 4(Dharmacon, T-2004-03) and Optimem (Gibco, 31985) for 24 hours, per the manufactures instructions.

Pooled APRIL siRNA (Mixed J-011523-05, J-011523-06, J-011523-07 and J-011523-08 (Dharmacon, Invitrogen, and Ambion), KSP positive control (custom made WANWK-000001) or negative control siRNA (On-Target Plus Duplex, Dharmacon, D-001810-10).

24 hours post transfection cells were harvested and plated as 5000 cell/100 in Black view plates (Perkin Elmer, 6005182) for functional assays, namely cell titer Glo (Promega, G7571) and Caspase Glo (Promega, G8093). Cells were also harvested to determine siRNA knockdown by Taqman and protein knockdown using an ELISA kit (BenderMedsystems, BMS2008).

Table 1 shows the effect of siRNA-mediated knockdown of endogenously expressed APRIL on epithelial cancer line proliferation. All cell lines were purchased from American Type Tissue Culture.

TABLE 1

| | Timepoint | Effect on proliferation |
|---|---|---|
| Cell Line | (hrs) | (% decrease) |
| PC3 -exp 1 | 48 | 30% |
| | 72 | 50% |
| PC3 -exp2 | 48 | 27% |
| | 72 | 10% |
| PC3 -exp 3 | 48 | 36% |
| | 72 | 33% |
| | 96 | 41% |
| PC3 -exp 4 | 48 | 63% |
| | 72 | 57% |
| | 96 | 43% |
| MALME 3M | 48 | 36% |
| | 72 | 20% |
| | 96 | 43% |

### III. APRIL Binding and its Inhibition: Flow Cytometry Staining

Cells were harvested, washed once with PBS, and resuspended as 1.5×10⁵ cells/150 μl. in PBS 0.5% BSA (Mediatech Inc 21031-CV, Sigma A3294). Cells were blocked with 100 μm/ml of Chromapure human IgG whole molecule (Jackson Immuno Research, 009-000-003) in PBS-BSA for 15 minutes on ice.

Subsequently cells were with incubated with APRIL-Fc or TRAILR2-Fc as a control at 1 ng/μl. Bound APRIL was detected using an anti-human IgG Fcγ (Jackson Immuno Research, 109-116-098) at 5 μg/ml.

For inhibition of binding experiments April was incubated with 100, 50, 25 and 12.5 μgm/ml rabbit anti-hu APRIL-HSPG pAb (Invitrogen, Zymed), or 16 and 8 μg/ml BCMA (Peprotech, 310-16) or antibody and BCMA in combination for 2 hours on ice prior adding to the blocked cells. The cells were then kept at 37 degrees for 30 minutes, washed with PBS-BSA and APRIL detected using an anti-human IgG Fcγ (Jackson Immuno Research, 109-116-098) at 5 μg/ml.

Table 2 shows that inhibiting binding of exogenous APRIL (an APRIL-Fc fusion protein generated in-house) through both the TNF-binding site (BCMA) and the HSPG-binding site (anti huAPRIL-HSPG pAb) inhibited APRIL binding to RPMI8226 cells better than either reagent alone. Data is expressed as geometric mean—fold over background, where the background is the secondary-only control. BCMA-Ig was at a concentration of 16 μg/ml, while the anti huAPRIL-HSPG pAb was at a concentration of 50 μg/ml.

TABLE 2

| | Fold |
|---|---|
| | binding over |
| | background |
| secondary only control | 1 |
| Binding of APRIL-Fc fusion protein | 14.83 |
| April-Fc fusion + Isotype control | 14.32 |
| April Fc + Anti April-HSPG pAb | 9.48 |
| April Fc + BCMA | 10.74 |
| April Fc + BCMA + Anti April-HSPG pAb | 7.35 |

### IV. MTT Assay to Measure Proliferation

RPMI 8226 cells (ATCC) were split at a density of 4×10⁵ cells/ml 1 day prior to assay. On the day of assay, 50 μl of cell suspension (20,000 cells/well) were plated onto a 96 well black view plate (Perkin-Elmer). 2× concentrations of the proteins listed in Table 3 (40 ug/ml TACI-Fc; 100 ug/ml anti-APRIL-HSPG pAb) were added. At 44 hours, cells were incubated with 10 μl of MTT reagent (Cell proliferation kit I, Roche cat #11465007001) for 4 hours. Cells were solubilized overnight in the incubator by adding the solubilization reagent (kit component). Plates were read at an absorbance of 550 nM and 690 nM. Aborbance is proportional to cell number and was used to calculate fold change from control.

Table 3 shows that inhibiting endogenous APRIL through both the TNF-binding site (TACI-Fc) and the HSPG-binding site (anti huAPRIL-HSPG pAb) inhibited proliferation of RPMI8226 cells better than either reagent alone.

TABLE 3

| | % reduction | Mean Value | |
|---|---|---|---|
| | relative to PBS | (Abs 550- | |
| Protein | control | 690 nM) | Std dev |
| PBS Ctrl | | 0.874 | 0.009 |
| Negative control Fc-fusion | 12.01 | 0.769 | 0.019 |
| protein | | | |
| TACI Fc | 33.18 | 0.584 | 0.003 |
| Anti huApril-HSPG pAb | 22.43 | 0.678 | 0.012 |
| TACI Fc + | 40.27 | 0.522 | 0.016 |
| Anti huApril-HSPG pAb | | | |

Table 4 shows anti-huAPRIL-HSPG pAb inhibited binding of APRIL to the cells in a dose-titratable manner Data was generated using binding protocol used for generating data in table 2. APRIL-Fc binding to the surface of hematopeoitic cancer (RPMI8226 and NM-2) and epithelial cancer (PC3 and HCT116) cell lines in the presence of no antibody was measured. The ability of an anti-APRIL-HSPG binding site pAb to inhibit this binding was evaluated in 2-fold titrations (12.5, 25, 50, 100 ug/ml). Matching concentrations of isotype control Ab were measured in parallel. While the isotype control Ab had very little impact on binding of exogenous APRIL, the anti-APRIL-HSPG pAb inhibited APRIL binding in a dose-titratable manner in all four cell lines. Data was calculated as Geometric mean PE Area: Fold over background.

TABLE 4

| cell line: | HCT116 | PC3 | RPMI8226 | JVM2 |
|---|---|---|---|---|
| secondary only control | 1.00 | 1.00 | 1.00 | 1.00 |
| irrelevant control Fc-fusion | 1.04 | 1.04 | 1.01 | 1.41 |
| protein | | | | |
| Binding of APRIL-Fc fusion | 13.90 | 16.52 | 16.70 | 27.41 |
| protein | | | | |
| Anti April-HSPG pAb | 1.07 | 1.04 | 0.94 | 1.11 |
| (100 μg/ml) | | | | |
| Isotype control (100 μg/ml) | 0.99 | 1.04 | 0.97 | 1.41 |
| April Fc + Anti April-HSPG | 4.13 | 3.72 | 3.92 | 6.44 |
| pAb (100 μg/ml) | | | | |
| April Fc + Isotype control | 10.10 | 9.67 | 12.97 | 24.87 |
| (100 μg/ml) | | | | |
| April Fc + Anti April-HSPG | 6.28 | 5.29 | 4.67 | 9.31 |
| pAb (50 μg/ml) | | | | |
| April Fc + Isotype control | 12.27 | 13.31 | 15.64 | 27.47 |
| (50 μg/ml) | | | | |
| April Fc + Anti April-HSPG | 10.14 | 6.87 | 8.39 | 15.38 |
| pAb (25 μg/ml) | | | | |
| April Fc + Isotype control | 12.76 | 13.30 | 16.22 | 28.33 |
| (25 μg/ml) | | | | |
| April Fc + Anti April-HSPG | 13.73 | 12.10 | 15.81 | 24.74 |
| pAb (12 μg/ml) | | | | |
| April Fc + Isotype control | 13.48 | 13.07 | 17.83 | 28.45 |
| (12 μg/ml) | | | | |

### V. APRIL Bispecific Antibody

The variable domain regions of an antibody contain sequences that form the antigen binding site, and a typical therapeutic IgG antibody has two identical antigen-binding variable regions that bind to a single target. Bispecific antibodies (BsAb) are antibodies that have two different variable domain regions and thus two different binding specificities within a single molecule. The dual binding specificities are a result of the variable domains binding to two different antigen-binding sites, or epitopes. These epitopes can either be on separate target molecules or on the same target.

The APRIL BsAb described in this invention binds to two separate epitopes on the same target, APRIL, corresponding to the receptor binding sites of the HSPG receptor and TACI/BCMA receptors. The HSPG receptor binds to a basic sequence (QKQKKQ) near the N-terminus of the mature human APRIL (Dillon, S. R. et al. (2006) Nature Rev. Drug Disc. 5:235-246). Both TACI and BCMA are TNFR that bind via a conserved DXL motif to the same hydrophobic pocket region on the APRIL trimer (Wallweber, H. J. A. et al. (2004) J. Mol. Biol. 343:283-290; and Hymowitz, S. G. et al. (2005) J. Biol. Chem. 280:7218-7227).

The BsAb described blocks binding of APRIL to HSPG receptor and to both TACI and BCMA, or to either TACI or BCMA, depending on the chosen epitope. The BsAb can bind directly to the HSPG and TACI/BCMA binding sites on APRIL to block receptor binding, or it can bind close to these sites such that APRIL is prevented from binding the receptors due to a steric hindrance effect.

The BsAb are produced using various formats including chemically joining together two independent antibodies or antibody fragments (Nisonoff et al. (1961) Arch. Biochem. Biophys. 93:460-464; and Cao, Y. et al. (2003) Adv. Drug. Deliv. Rev. 55:171-197), fusing together hybridomas to make quadromas (Cao, et al. (2003) supra), or by using DNA engineering to rationally design the BsAb (Cao, et al. (2003) supra). Engineered BsAb are customized for such characteristics as size, valency, serum half-life, affinity and effector functions.

The described BsAb are engineered to contain an Fc domain if effector functions and long serum half-life are required. The Fc domain are also engineered to modify the effector functions and half-life (Shields, R. L. et al. (2002) J. Biol. Chem. 277(30):26733-26740). Such BsAb can be generated using a knobs-into-holes method of altering amino acid side chains in the CH3 domain to produce heavy chains that will preferentially heterodimerize (Merchant, M. A. et al. (1998) Nat. Biotechnol. 16:677-681; and Xie, Z. et al. (2005) J. Immunol. Meth. 296:95-101). These BsAb require a common light chain for both heavy chains unless the antibody is designed using a heavy chain similar to or including those produced by camels and llamas, which do not use a light chain (Conrath, K. E. et al. (2001) J. Biol. Chem. 276 (10):7346-735; and Shen, J. et al. (2007) J. Immunol. Meth. 318:65-74). Alternatively, two different single chain antibody fragments (scFv) containing only the antigen binding variable domains of the heavy and light chain artificially linked together by a flexible polypeptide can be substituted for a portion of the heavy chain including the variable domain (Cao, et al. (2003) supra; and Hudson, P. J. et al. (1999) J. Immunol. Meth. 231:177-189). A combination of one full heavy chain/light chain can associate with a second heavy chain-scFv to produce a BsAb. These Fc-containing BsAb are similar in size to a typical full-length IgG antibody (˜150 kDa), and are produced using standard mammalian tissue culture methods.

If effector functions are not required and smaller size is desired for possible improved tissue penetrance, BsAb lacking the Fc domain are produced. Antibodies lacking the Fc domain will have a shorter serum half-life, which may also be desired. Antibody fragments such as different scFv or Fab domains, a combination of both scFv and Fab domains (Schoonjans, R. et al. (2000) J. Immunol. 165:7050-7057), or alternatively single heavy chain antibodies and fragments similar to and including antibodies produced by camels and llamas (Conrath, et al. (2001) supra; Shen, J. et al. (2007) supra), can be joined together using multiple approaches. Antibody fragments can be directly joined together by a flexible peptide linker, or can be fused to the heavy chain CH3 domain and joined together by the knobs-into-holes method. Different antibody fragments can also be joined together to form BsAb by fusing them to domains that preferentially heterodimerize, such as the fos and jun leucine zipper motifs (Cao, et al. (2003) supra). Sizes of these BsAb would range from ˜60 kDa to ˜100 kDa. Depending on the construct, these BsAbs would be produced in either mammalian cells or within microbial hosts.

The described BsAb can also have multiple valency for one or both of the epitopes targeted on APRIL. Multiple valency can increase functional binding affinity to the target by increasing binding avidity. scFv fragments can be engineered to form dimers (diabody, ˜60 kDa), trimers (triabody, ˜90 kDa) and tetramers (tetrabodies, ˜120 kDa) simply by adjusting the length and flexibility of the polypeptide linker between the VH/VL of a scFv and the linker joining two scFv (Hudson, P. J. et al. (1999) J. Immunol. Meth. 231:177-189). Alternatively, Fab domains are linked together to form multivalent binding (Miller, K. et al. (2003) J. Immunol. 170:4854-4861). Bispecific triabodies and tetrabodies are designed to have monomeric binding against one epitope and bivalent or trivalent binding against another epiotpe. Bispecific tetrabody can also be designed to have bivalent binding to both epitopes on APRIL.

Combinations of antibodies and antibody fragments are joined together to produce BsAb with multiple valencies. For example, a Fab targeting one epitope on APRIL can have scFv targeting the other epitope on APRIL linked to both the heavy and light chains of the Fab, resulting in multivalent binding to the second epitope. Similarly, a F(ab′)2, or multiple linked Fab domains targeting one epitope can have scFv linked to the light chains or to a heterodimerizing domain to produce a BsAb with bivalency against each epitope. Similar BsAb can be produced with full Fc-containing bivalent antibodies, if size is not an issue.

### VI. Binding of Anti-HSPG mAb (124) to APRIL Fc

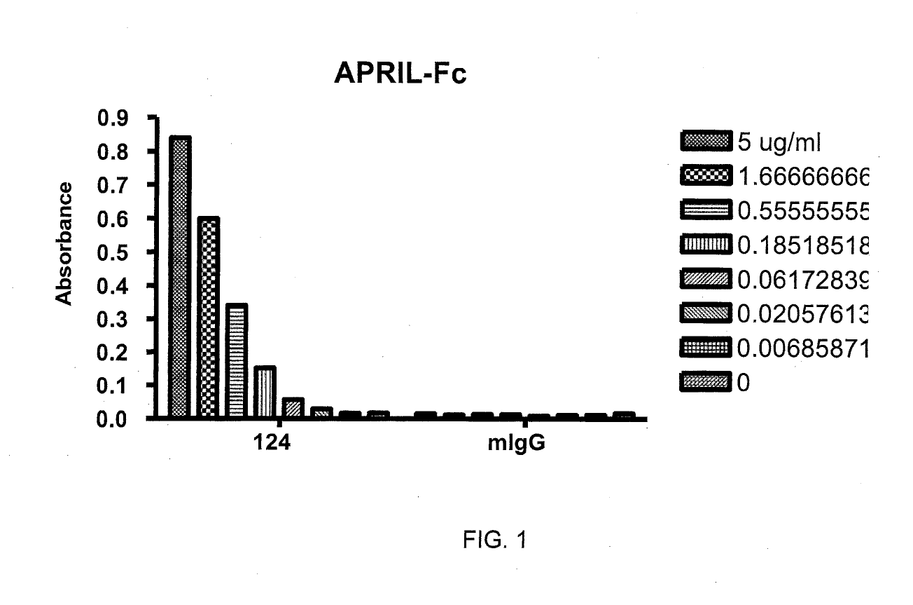

Goat anti-human IgG, Fab2 fragment was coated onto Nunc MaxiSorp plates at 0.5 ug/ml in PBS at 4 C overnight. Plates were washed and blocked with PBS containing 5% BCS for 1 hour at room temperature. An hIgG1 Fc-hAPR1L fusion protein was captured onto the plate by adding conditioned media from 293F cells transiently transfected with the pYD5-IgSP-hIgG1Fc-TEV-hAPRIL construct. Conditioned media was diluted 1:4 with PBS containing 1% BCS and added to the ELISA plates for 1 hour at room temperature. An antibody (124) to the APRIL HSPG binding site or control antibodies were diluted to 5 ug/ml in PBS containing 1% BCS and serially diluted 3-fold to approximately 0.007 ug/ml. Diluted antibody was added to ELISA plates with captured APRIL and incubated for 1 hour at room temperature. Bound antibody was detected using a goat anti-mouse IgG-HRP secondary antibody (Southern Biotech) diluted 1:4000 in PBS/1% BCS. The secondary antibody was incubated for 1 hour at room temperature and plates were developed with TMB (BD Biosciences) and stopped with 0.5M sulfuric acid. The absorbance at 450 nm-620 nm was determined. FIG. 1 shows binding of 124 to the APRIL-Fc protein in a concentration-dependent manner.

### VII. HSPG Peptide Binding by Anti-HSPG Antibody

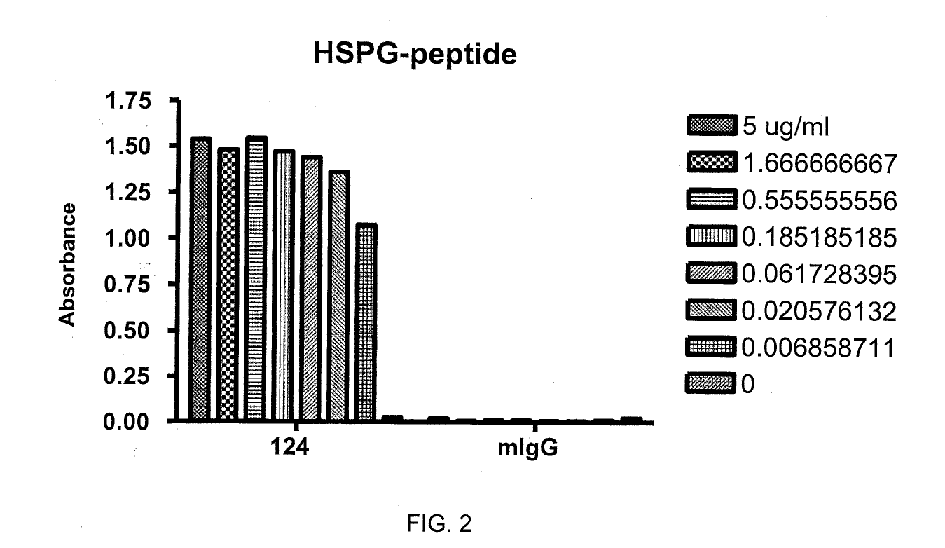

A peptide corresponding to the N-terminal HSPG binding region of APRIL AVLTQKQKKQSAAAC was synthesized by Anaspec. Unconjugated peptide was coated onto Nunc Maxisorp plates at 1 ug/ml in PBS at 4 C overnight. Plates were washed and blocked with PBS containing 5% BCS for 1 hour at room temperature. An antibody against the HSPG site of APRIL (124) or control antibodies were diluted to 5 ug/ml in PBS containing 1% BCS and serially diluted 3-fold to 0.007 ug/ml. Diluted antibody was added to the peptide coated plates and incubated for 1 hour at room temperature. Bound antibody was detected using goat anti-mouse IgG-HRP (Southern Biotech) diluted 1:4000 in PBS/1% BCS. The secondary antibody was incubated for 1 hour at room temperature, and plates were developed with TMB (BD Biosciences) and stopped with 0.5M sulfuric acid. The absorbance at 450 nm-620 nm was determined. FIG. 2 shows binding of 124 to the HSPG peptide at various concentrations.

### VIII. HSPG mAb (124) Does not Compete with BCMA Binding to APRIL

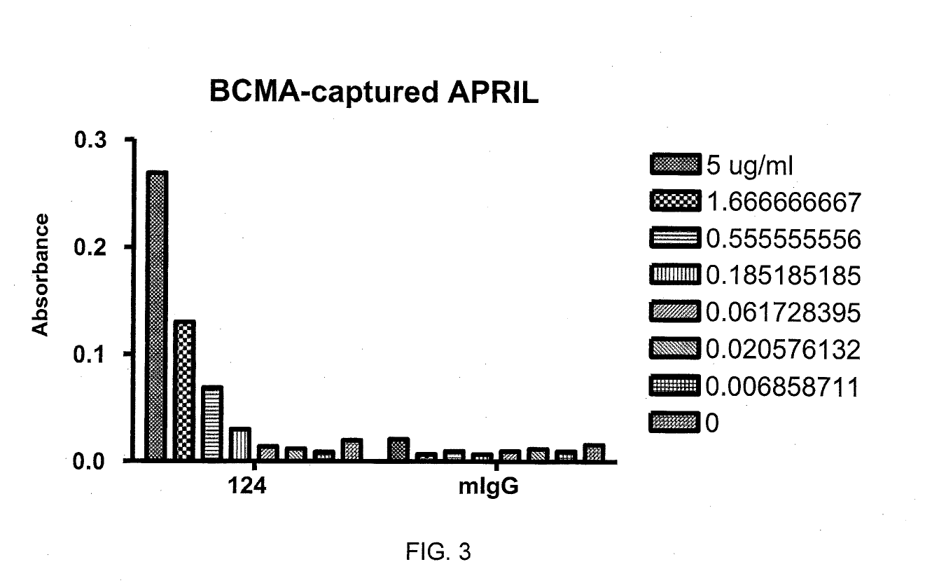

Recombinant BCMA (Peprotech) was coated onto Nunc Maxisorp plates at 0.5 ug/ml in PBS at 4 C overnight. Plates were washed and blocked with PBS containing 5% BCS for 1 hour at room temperature. Mature, secreted APRIL was captured onto the plate by adding conditioned media from 293F cells transiently transfected with full-length APRIL cDNA. Conditioned media was diluted 1:3 with PBS containing 1% BCS and added to the ELISA plates for 1 hour at room temperature. The 124 mAb or control antibodies were diluted to 5 ug/ml in PBS containing 1% BCS and serially diluted 3-fold to 0.007 ug/ml. Diluted antibody was added to ELISA plates with captured APRIL and incubated for 1 hour at room temperature. Bound antibody was detected using goat anti-mouse IgG-HRP (Southern Biotech) diluted 1:4000 in PBS/1% BCS. The secondary antibody was incubated for 1 hour at room temperature and plates were developed with TMB (BD Biosciences) and stopped with 0.5M sulfuric acid. The absorbance at 450 nm-620 nm was determined. FIG. 3 shows that the 124 mAb does not compete with BCMA binding of APRIL.

### IX. Polyclonal HSPG Antibody Blocks Binding of 124 mAb to APRIL

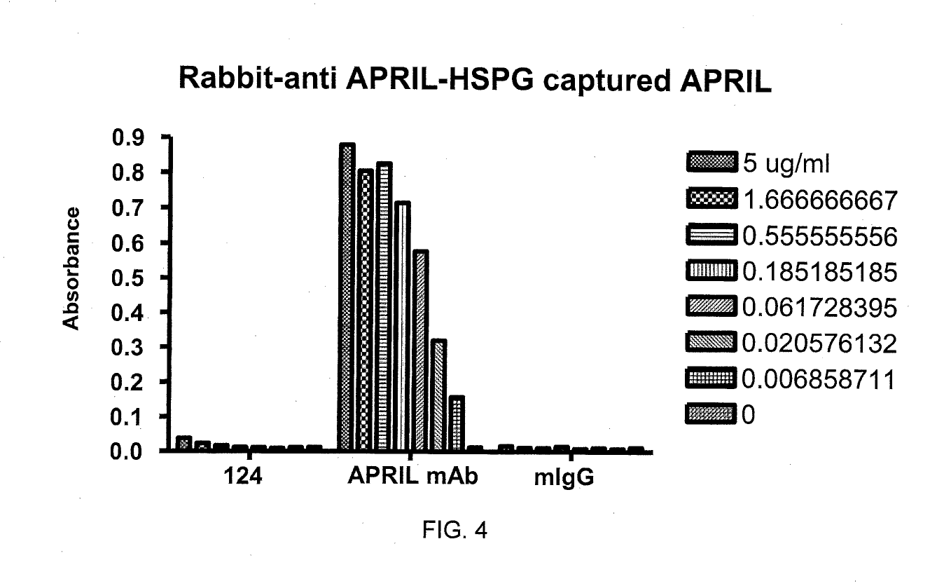

A polyclonal rabbit anti-APRIL antibody specific for the HSPG region of APRIL was generated by immunizing rabbits with the HSPG peptide conjugated to KLH, AVLTQKQKKQSAAAC-KLH. This antibody was coated onto Nunc Maxisorp plates at 1 ug/ml in PBS at 4C overnight. Plates were washed and blocked with PBS containing 5% BCS for 1 hour at room temperature. Mature, secreted APRIL was captured onto the plate by adding conditioned media from 293F cells transiently transfected with full-length APRIL cDNA. Conditioned media was diluted 1:3 with PBS containing 1% BCS and added to the ELISA plates for 1 hour at room temperature. The 124 mAb or control antibodies were diluted to 5 ug/ml in PBS containing 1% BCS and serially diluted 3-fold to 0.007 ug/ml. Diluted antibody was added to ELISA plates with captured APRIL and incubated for 1 hour at room temperature. Bound antibody was detected using rabbit anti-mouse IgG-HRP (Jackson Immunoresearch Labs) diluted 1:4000 in PBS/1% BCS. The secondary antibody was incubated for 1 hour at room temperature, and plates were developed with TMB (BD Biosciences) and stopped with 0.5M sulfuric acid. The absorbance at 450 nm-620 nm was determined. FIG. 4 shows that the polyclonal HSPG antibody blocks binding of 124 to APRIL.

### X. HSPG mAb Inhibits Proliferation of B Cell Lymphoma Cells

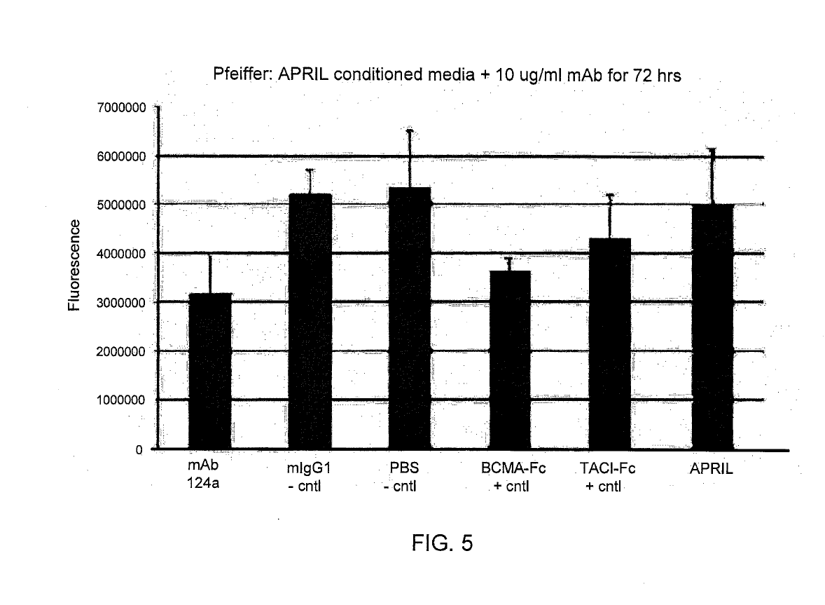

10,000 B cell lymphoma (Pfeiffer) cells/well were treated with conditioned media with or without the 124 mAb, control IgG1 mAbs, BCMA-Fc protein, or TACI-Fc protein. Antibodies were preincuabted with the conditioned media for 30 minutes at room temperature prior addition to the cells. After 72 hours added 10 μl Alamar blue reagent to the wells. Fluorescence read after 4 hours with a plate reader. Data plotted as RLU after subtracting from the blank wells. FIG. 5 shows inhibition of Pfeiffer cell proliferation in the presence of 124, BCMA-Fc, or TACI-Fc.

1. A method of inhibiting a cancer by administering at least one APRIL antagonist, wherein the antagonist binds to at least two distinct epitopes on the APRIL polypeptide, wherein the epitopes comprise a binding site for BCMA or TACI, and a binding site for HSPG.

2. The method of claim 1, wherein a first and a second APRIL antagonist are administered.

3. The method of claim 2, wherein the first APRIL antagonist is an antibody or fragment thereof, that binds to the binding site for BCMA or TACI; and the second APRIL antagonist is an antibody or fragment thereof, that binds to the HSPG binding site.

4. The method of claim 2, wherein the first APRIL antagonist comprises a soluble BCMA protein or a soluble TACI protein, and the second APRIL antagonist comprises an antibody or fragment thereof that binds to the HSPG binding site.

5. The method of claim 1, wherein the APRIL antagonist is a bispecific antibody, or fragment thereof, that binds to the BCMA or TACI binding site and the HSPG binding site.

6. The method of claim 5 wherein the bispecific antibody or fragment thereof is a humanized or fully human antibody.

7. A method of inhibiting a B cell disorder by administering at least one APRIL antagonist, wherein the antagonist binds to at least two distinct epitopes on an APRIL polypeptide, wherein the epitopes comprise a binding site for BCMA or TACI, and a binding site for HSPG.

8. The method of claim 7, wherein a first and a second APRIL antagonist are administered.

9. The method of claim 8, wherein the first APRIL antagonist comprises an antibody or fragment thereof, binds to the binding site for BCMA or TACI; and the second APRIL antagonist comprises an antibody or fragment thereof, that binds to the HSPG binding site.

10. The method of claim 8, wherein the first APRIL antagonist comprises a soluble BCMA protein or a soluble TACI protein, and the second APRIL antagonist comprises an antibody or fragment thereof that binds to the HSPG binding site.

11. The method of claim 7, wherein he APRIL antagonist is a bispecific antibody, or fragment thereof, that binds to the BCMA or TACI binding site, and the HSPG binding site.

12. The method of claim 11 wherein the bispecific antibody or fragment thereof is a humanized or fully human antibody.

13. A method of inhibiting a cancer or a B cell disorder by administering an APRIL antagonist, wherein the APRIL antagonist is a humanized or fully human antibody or fragment thereof, and inhibits APRIL binding to HSPG by binding to an epitope on APRIL comprising an HSPG binding site
